(19) United States
(12) Patent Application Publication
(10) Pub. No.: US 2011/0020241 A1
(43) Pub. Date: Jan. 27, 2011

(54) FLUORESCENT LABELING AGENT CONTAINING QUANTUM DOTS

(75) Inventors: Kazuya Tsukada, Kanagawa (JP); Kazuyoshi Goan, Kanagawa (JP); Tuneko Fujii, Tokyo (JP)

Correspondence Address: LUCAS & MERCANTI, LLP, 475 PARK AVENUE SOUTH, 15TH FLOOR, NEW YORK, NY, 10016, US

(73) Assignee: KONICA MINOLTA MEDICAL & GRAPHIC, INC., Tokyo (JP)

(21) Appl. No.: 12/934,732

(22) PCT Filed: Mar. 4, 2009
(86) PCT No.: PCT/JP2009/054057

§ 371 Date: Sep. 27, 2010

(30) Foreign Application Priority Data
Aug. 6, 2008 (JP) 2008202865
Aug. 6, 2008 (JP) 2008202866

(51) Int. Cl. A61K 49/00

(52) U.S. Cl. 424/9.6; 977/774

(57) ABSTRACT

Disclosed is a fluorescent labeling agent containing quantum dots, which exhibits little fluctuation in luminous properties in accordance with the environment where the fluorescent labeling agent is stored and therefore has high environmental stability, and which has high luminous intensity. Specifically disclosed is a fluorescent labeling agent containing quantum dots, which comprises: (1) a fluorescent labeling agent core part which comprises at least three quantum dots per a fluorescent labeling agent and a protective material; and (2) an organic surface-coating layer which coats the fluorescent labeling agent core part, wherein the fluorescent labeling agent core part has an average particle diameter of 10 to 50 nm.

### TECHNICAL FIELD

The present invention relates to a fluorescent labeling agent containing a quantum dot which has high luminous intensity and high environmental stability.

### BACKGROUND

Recent advancement in nanotechnology indicates the possibility that inorganic nanoparticles can be used in detection, diagnosis, sensing, and other applications. Further, over recent years, inorganic nanoparticles, which mutually interact with biological systems, have attracted wide attention in the biological and medical fields. These inorganic nanoparticles are thought to be promising as novel intravascular probes for both sensing (for example, imaging) and medical treatment purposes (for example, drug delivery).

Incidentally, in an inorganic nanoparticle, a nanoparticle comprising a material, which is a semiconductor material of nanometer size, producing a quantum confinement effect is referred to as “a quantum dot.” Such a dot is a tiny agglomerate of at most ten-odd nm composed of several hundreds to several thousands of aggregated semiconductor atoms, and emits energy corresponding to the energy band gap of the quantum dot when reaching an energy excitation state via absorption of light from an excitation source.

Accordingly, when the size or material composition of the quantum dot is adjusted, the energy band gap can be controlled, whereby energy can be utilized as light at various wavelength bands. Therefore, recently a development of technology in the field of biology and medical is expected, in which the semiconductor nanoparticle is applied to a labeling agent for obtaining various information about chemical materials and molecules constituting a living cell.

As a labeling agent for detecting a biological molecule, employed are an organic dye, fluorescent protein and an inorganic nanoparticle such as gold nanoparticle or CdSe which has surface modified functional group needed for adapting for dispersing in a living body.

Fluorescent agent is preferably used which has high detectability by efficient use of photon. Among them, in the view of stability for photobleaching, inorganic fluorescent agent is usable. As an inorganic fluorescent agent, an inorganic fluorescent particle is used and particle size is contrived to be 50 nm or less (nanoparticle) in terms of adapting a vital observation.

As conventional methods for preparing nanoparticle, various method including liquid phase method, gas phase method and solid phase method are investigated. However, in each method, an influence of lattice strain and lattice defect of particle surface caused by nanosizing increases according to increasing of surface area, to result in producing problem of deterioration of luminous intensity, especially producing mortal deterioration of luminous intensity in the case of detection in aqueous medium (for example, refer to Patent Document 1 and 2). Further, it has a problem of fluctuation in luminous properties in accordance with the stored environment (pH, temperature).

Patent Document 1: Japanese Patent Application Publication No. 3636970

Patent Document 2: Japanese Patent Application Publication No. 3771925

### DISCLOSURE OF THE INVENTION

### Problems to be Solved by the Invention

In view of the foregoing, the present invention was achieved. An object of the present invention is to provide a fluorescent labeling agent containing quantum dots, which exhibits little fluctuation in luminous properties in accordance with the environment, namely has high environmental stability, and which has high luminous intensity.

### Means to Solve the Problems

The above problems can be solved via the following means:

1. A fluorescent labeling agent containing quantum dots comprising: (1) a fluorescent labeling agent core part which comprises at least three quantum dots per a fluorescent labeling agent and a protective material; and (2) an organic surface-coating layer which coats the fluorescent labeling agent core part, wherein the fluorescent labeling agent core part has an average particle diameter of 10 to 50 nm.

2. The fluorescent labeling agent of item 1, wherein the quantum dots comprising silicon (Si).

3. The fluorescent labeling agent of item 1, wherein the quantum dots comprises activated fluorescent nanoparticles, near-infrared fluorescent nanoparticles emitting near-infrared light having a wavelength in the range of 700-2,000 nm, at least a part of composition of the quantum dots is represented by Formula (1): APO₄, or Formula (2): AF₃ (wherein, A is an atom selected from yttrium (Y), lutetium (Lu) and lanthanum (La), and comprising a rare earth element as an activator.

4. The fluorescent labeling agent of item 3, wherein the rare earth element is any one or combination of praseodymium (Pr), neodymium (Nd), holmium (Ho), erbium (Er) or ytterbium (Yb).

5. The fluorescent labeling agent of any one of items 1 to 4, wherein the protective material is SiOₓ, wherein x=1.5-2.0, or ZnS.

6. The fluorescent labeling agent of any one of items 1 to 5, wherein a number of the quantum dots per one fluorescent labeling agent is 3-10.

7. The fluorescent labeling agent of any one of items 1 to 6, wherein the organic surface-coating layer is chemically-modified by a compound represented by Formula (PEG):

X—(CH₂CH₂O)ₙ—Y, Formula (PEG)

wherein X represents a linkage group containing a linking group at terminal linkable to a compound constituting the organic surface-coating layer, Y represents hydroxyl group, alkoxy group, and a functional group at terminal bondable to a biomolecule, and n represents an integer of 1-20.

### EFFECTS OF THE INVENTION

The present invention made it possible to provide a fluorescent labeling agent containing quantum dots, which exhibits little fluctuation in luminous properties in accordance with the stored environment and therefore has high environmental stability, and which has high luminous intensity.

### DESCRIPTION OF THE PREFERRED EMBODIMENT

A fluorescent labeling agent related to the present invention is characterized in containing quantum dots which comprises (1) a fluorescent labeling agent core part which comprises at least three quantum dots per fluorescent labeling agent and a protective material; and (2) an organic surface-coating layer which coats the fluorescent labeling agent core part, wherein the fluorescent labeling agent core part has an average particle diameter of 10 to 50 nm. This characteristic is common technical characteristic in the invention related to claims 1 to 7.

As the preferred embodiment of the present invention, the quantum dots comprise silicon (Si).

Further, the preferred embodiment is that the quantum dots comprises activated fluorescent nanoparticles, near-infrared fluorescent nanoparticles emitting near-infrared light having a wavelength in the range of 700-2,000 nm, at least a part of composition of the quantum dots is represented by Formula (1): APO₄, or Formula (2): AF₃ (wherein, A is an atom selected from yttrium (Y), lutetium (Lu) and lanthanum (La), and comprises a rare earth element as an activator. Herein, preferred is that the rare earth element is any one or combination of praseodymium (Pr), neodymium (Nd), holmium (Ho), erbium (Er) or ytterbium (Yb).

According to the present invention, preferred is that the protective material is SiOₓ, wherein x=1.5-2.0, or ZnS. Further, preferred is that a number of the quantum dots per one fluorescent labeling agent are 3-10.

As the preferred embodiment of the present invention, the organic surface-coating layer is chemically-modified by a compound represented by Formula (PEG):

X—(CH₂CH₂O)—Y, Formula (PEG)

wherein X represents a linkage group containing a linking group at terminal to a compound constituting the organic surface-coating layer, Y represents hydroxyl group, alkoxy group, and a functional group at terminal bondable to a biomolecule, and n represents an integer of 1-20.

Constituent element and best mode/embodiments of the present invention will now be specifically detailed.

(Quantum Dot)

A fluorescent labeling agent related to the present invention is characterized in containing quantum dots which comprises (1) a fluorescent labeling agent core part which comprises at least three quantum dots per fluorescent labeling agent and a protective material; and (2) an organic surface-coating layer which coats the fluorescent labeling agent core part, wherein the fluorescent labeling agent core part has an average particle diameter of 10 to 50 nm. Further, preferred is that a number of the quantum dots per one fluorescent labeling agent are 3-10.

As a material for quantum dots related to the invention, various conventional fluorescence emitting compound and its raw material can be employable. For example, other than semiconductor material described later, rare earth element such as erbium (Er), holmium (Ho), praseodymium (Pr), thulium (Tin), neodymium (Nd), gadolinium (Gd), europium (Eu), ytterbium (Yb), samarium (Sm) and cerium (Ce); and halogenated compound containing thereof can be employable.

Semiconductor nanoparticle below is preferably employed as quantum dots of the present invention.

<Semiconductor Nanoparticles>

As materials of semiconductor nanoparticles according to the present, conventional fluorescence emitting compound and its raw material can be employable. For example, various semiconductor materials known as materials of semiconductor nanoparticles can be formed. Specifically, semiconductor compounds of group IV, groups II-VI, and groups III-V in the periodic table of the elements and raw material compound containing element of constituting these compounds can be used.

Of semiconductors of groups II-VI, there are specifically listed MgS, MgSe, MgSe, MgTe, CaS, CaSe, CaTe, SrS, SrSe, SrTe, BaS, BaSe, BaTe, ZnS, ZnSe, ZnTe, CdS, CdSe, HgS, HgSe, and HgTe.

Of semiconductors of groups III-V, there are preferable GaAs, GaN, GaPGaSb, InGaAs, InP, InN, InSb, InAs, AlAs, AlP, AlSb, and AIS.

Of semiconductors of group IV, Ge and Si are specifically suitable.

Among these various semiconductor materials, in view of a composition satisfying safety, preferred are Si, Ge, InN, and InP. Of theses, silicon (Si) and gelmanium (Ge) are specifically preferable as a main component element constituting semiconductor nanoparticle of the present invention. According to the present invention, “main component element constituting semiconductor nanoparticle” means an element which has maximum content ratio among the elements constituting the semiconductor nanoparticle.

In the present invention, a semiconductor nanoparticle is preferably formed as a particle having a core/shell structure. In this case, such a semiconductor nanoparticle is a so-called core/shell type semiconductor particle having a core/shell structure which is structured of a core particle composed of a semiconductor nanoparticle and a shell layer covering the core particle. The core particle and the shell layer each preferably differ in chemical composition. Thereby, a band gap of shell is preferably higher than that of core.

Shell is necessary to stabilize a surface defect of core particle and to enhance luminance, and also important so as to form a surface on which surface modifying agent can easily be absorbed and bonded. With respect to the effect of the present invention, it is an important constitution which enhances an accuracy of detection sensitivity.

The core particle and the shell layer will now be described.

<Core Particle>

As semiconductor materials used for a core particle, various types of semiconductor materials can be employed. There are specifically listed, for example, MgS, MgSe, MgTe, CaS, CaSe, CaTe, SrS, SrSe, SrTe, BaS, BaTe, ZnS, ZnSe, ZnTe, CdS, CdSe, CdTe, GaAs, GaP, GaSb, InGaAs, InP, InN, InSb, InAs, AlAs, AlP, AlSb, AlS, PbS, PbSe, Ge, and Se, or mixtures thereof. In the present invention, a specifically preferable semiconductor material is Si.

The quantum dot (semiconductor nanoparticle) of the present invention is characterized to have fluorescent emission in the range of 350 nm-1,100 nm. In view of preventing an influence of emission from living cell itself and enhancing SN ratio, the quantum dot emits in a wavelength region of from near infrared to infrared region. Thus, average diameter of core is preferably adjusted to emit infrared emission. Therefore, average diameter of core is preferably 0.5-1.5 nm, more preferably 1-5 nm.

Herein, average diameter of semiconductor nanoparticle has to be evaluated exactly by three dimensions, however it is difficult due to too fine particles. Therefore, actually, evaluation has to be carried out by two dimension image, preferred is that many electron microscope photo images of different scenes are took by using transmission electron microscope (TEM) and diameter is calculated by averaging of many partickes. Preferable numbers of particles took by TEM have to be 100 or more.

<Shell Layer>

As semiconductor materials used for a shell, various types of semiconductor materials can be employed. There are specifically listed, for example, ZnO, ZnS, ZnSe, ZnTe, CdO, CdS, CdSe, CdTe, MgS, MgSe, GaS, GaN, GaP, GaAs, GaSb, InAs, InN, InP, InSb, AlAs, AlN, AlP, and AlSb, or mixtures thereof.

In the present invention, specifically preferable semiconductor materials are SiO₂ and ZnS.

Shell related to the present invention may not completely cover all surface of core particle, unless partially exposed core particle brings negative effect.

(Dopant)

The semiconductor nanoparticle of the present invention preferably contains different element having an equivalent valence electron configuration as main element constituting thereof or atom pair of foresaid different element as dopant, and forsaid dopant uniformly distributes on the surface or in the neighborhood of the semiconductor nanoparticle.

Herein, “valence electron” means the electron being in the outer most shell of the electron shells (K shell, L shell, M shell, . . . ) which constitutes element. Therefore, when silicon (Si) is used as a main component element constituting the semiconductor nanoparticle, four valence electrons are configured at the outer most shell, as for an element or atom pair having an equivalent valence electron configuration as main element constituting thereof, listed are Be—Be (Be pair), Mg—Mg (Mg pair) and Ge.

When silicon (Si) or germanium (Ge) is used as a main component element constituting the semiconductor nanoparticle, Be—Be is specifically preferred as dopant.

According to the present invention, dopant is necessary to be contained at the surface or its neighborhood of the semiconductor nanoparticles for its position. Herein, “neighborhood of surface” is within the range of 30% of its radius from the surface of the semiconductor nanoparticles, most preferably within the range of 15%.

Distribution state of the dopant related to the present invention can be observed and measured by analytical method using X-ray photoelectron spectroscopy (XPS/ESCA; XPS: X-ray Photoelectron Spectroscopy/ESCA: Electron Spectroscopy for Chemical Analysis). X-ray photoelectron spectroscopy is a method for observing solid surface and neighborhood thereof (for example, element composition) by measuring kinetic energy of electron ejected by irradiation of monochromatic ray (X-ray).

<Particle Size of Semiconductor Nanoparticle>

An average particle diameter of inorganic nanoparticle related to the present invention, for example, semiconductor nanoparticle is characterized to be 1 to 10 nm, preferably 1 to 5 nm.

In semiconductor nanoparticle related to the present invention, it is known that nano size particle having smaller particle size than a wavelength of electron (about 10 nm) exhibits specific physical property different from bulk body due to quantum size effect which increases size finite influence on movement of electron. Generally, a material, which is a semiconductor material of nanometer size, producing a quantum confinement effect, is referred to as “a quantum dot.” Such a dot is a tiny agglomerate of at most ten-odd nm composed of several hundreds to several thousands of aggregated semiconductor atoms, and emits energy corresponding to the energy band gap of the quantum dot when reaching an energy excitation state via absorption of light from an excitation source. Accordingly, when the size or material composition of the quantum dot is adjusted, the energy band gap can be controlled, whereby energy can be utilized at various wavelength bands. Further, quantum dots, namely semiconductor nanoparticle, are characterized in that emission wavelength can be controlled by changing the particle diameters even when the compositions are the same.

Semiconductor nanoparticles related to the present invention are controlled to emit fluorescence in the range of 350 to 1,100 nm. According to the present invention, emission of wavelength in near-infrared region is preferably used due to enhance SN ratio by preventing an influence of emission from living cell itself.

(Production Method of Semiconductor Nanoparticles)

To produce the semiconductor nanoparticle of the present invention, various types of methods such as liquid phase method or gas phase method conventionally known in the art can be employed.

Production methods employing a liquid phase method include a precipitation method, a coprecipitation method, a sol-gel method, a homogeneous precipitation method, and a reduction method. In addition, a reverse-micelle method and a supercritical hydrothermal synthesis method are excellent methods to produce nanoparticles (for example, refer to JP-A Nos. 2002-322468, 2005-239775, 10-310770, and 2000-104058).

In the case of producing semiconductor nanoparticles by liquid phase method, preferred is a method in which reduction process of precursors of the semiconductor nanoparticles by reduction reaction is included. Further, preferred is an embodiment in which a reaction of the precursors of the semiconductors is carried out under the existence of surfactant. Herein, the precursor of the semiconductor is a compound containing an element used in the semiconductor material. In the case of semiconductor being silicon (Si), the precursor of the semiconductor includes SiCl₄, for example. Other precursor of the semiconductor include InCl₃, P(SiMe₃)₃, ZnMe₂, CdMe₂, GeCl₄ and tributylphosphine selene.

Reaction temperature of the precursor is not limited so long as being higher than boiling point of the precursor of the semiconductor and lower than boiling point of solvent, without being restricted thereto, preferably in the range of 70 to 110° C.

(Reducing Agent)

As a reducing agent for reducing the precursor of the semiconductor, various types of reducing agents conventionally known in the art can be employed by selecting according to a condition. According to the present invention, in view of strength of reduction, preferred is reducing agent such as lithium aluminum hydride (LiAlH₄), sodium boron hydride (NaBH₄), sodium aluminium bis(2-methoxyethoxy) hydride, lithium boron tri(sec-butyl) hydride (LiBH(sec-C₄H₉)₃), potasium boron tri(sec-butyl) hydride, and lithium boron triethyl hydride. Specifically, preferred is lithium aluminum hydride (LiAlH₄) in view of strength of reduction.

(Solvent)

As a solvent for dispersing the precursor of the semiconductor, various types of solvents conventionally known in the art can be employed. Preferred are alcohols such as ethylalcohol, sec-butylalcohol, and t-butylalcohol; and hydrocarbon solvents such as toluene, decane and hexane. According to the present invention, hydrophobic solvent such as toluene is preferred as to dispersing solvent.

(Surfactant)

As a surfactant, various types of surfactants conventionally known in the art can be employed such as anionic, nonionic, cationic and ampholytic surfactant. Among them, preferred is tertiary ammonium salts such as tetrabutyl ammonium chloride, tetrabutyl ammonium bromide, hexafluoro phosphate, tetraoctyl ammonium bromide (TOAB), or tributylhexadecyl phosphonium bromide. Specifically, tetraoctyl ammonium bromide is preferred.

Reaction by liquid phase method is largely influenced by a state of compound including solvent in liquid. Attention is needed especially in the case of producing nano size particles exhibiting excellent monodisperse distribution. For example, in a reverse-micelle method, condition for forming nanoparticles is limited because size or state of the reverse-micelle as reaction field is changed by concentration or type of surfactant. Therefore, it is necessary to combine adequate surfactant and solvent.

As a gas phase method, employed are (1) a method in which raw semiconductor material is evaporated by the first high temperature plasma generated between opposed electrodes and passed into the second high temperature plasma generated by non-electrode discharge in a ambient of reduced pressure (for example, JP-A No. 6-2709015), (2) a method in which nanoparticles are separated and eliminated from an anode comprising raw semiconductor by electrochemical etching (for example, JP-A No. 2003-515459), (3) a laser abrasion method (for example, JP-A No. 2004-356163), and (4) a high speed sputtering method (for example, JP-A No. 2004-296781). Further, a method in which raw gas is reacted at low pressure in gas phase to synthesize powder including particles is preferably employed.

(Post Treatment after Preparation of Semiconductor Nanoparticles)

According to the method of preparation for semiconductor nanoparticles related to the present invention, the preferred embodiment includes any one of post treatment of plasma, heating, radiation or ultrasonic, after preparation of semiconductor nanoparticles, especially after forming shell.

As plasma treatment, in view of particle composition, crystallinity or surface, adequate process is selected from low/high temperature plasma, microwave plasma, atmospheric pressure plasma. Of these, microwave plasma is preferable.

As heat treatment, heat is applied by selecting ambient from any one of atmospheric, vacuum, or inert gas. Applied temperature range is different by a constitution of fluorescent particles. In the case of excessively high temperature, strain or peel may occur between core and shell. In the case of low temperature, effect of heat treatment becomes poor. Therefore, temperature of 100° C. or more and 300° C. or less is preferably employed.

As radiation treatment, employed are X-ray, γ ray, and neutron my having high energy; and vacuum ultraviolet ray (VUV), ultraviolet ray or short pulse laser while having low energy. Treatment time differs by type of radiation. In the case of X-ray, treatment time may be relatively short for any composition due to its high transmittance. In the case of ultraviolet, relatively long treatment time is needed.

Principle effect of these post treatment can not be solved yet, but estimated to enhance adhesion of interface between core and shell in core/shell type particle, to accelerate passivation, resulting in enhancing emission efficiency. Estimated is that influence appears remarkably in infrared luminous body, reflecting its performance.

According to the present invention, band gap of shell is preferably higher than that of core. Shell is necessary to stabilize a surface defect on core particle and to enhance luminance, and also important to form a surface on which surface modifying agent can easily be absorbed and bonded to be fluorescent labeling agent.

(Protective Material)

As a protective material related to the present invention, a material such as organic polymer or inorganic material which does not injure performance of quantum dot being protective object is employed. Preferably employed is semiconductor or oxide having band gap higher than that of quantum dot.

Specific example of preferable semiconductor as protective material includes ZnS to exhibit higher effect of quantum confinement of quantum dot. For example, in the case of Si being main composition of quantum dot, oxide SiO₂ can be employable as protective material. In the case of Ge being main composition of quantum dot, GeO₂ can be employable as protective material.

(Activated Fluorescent Nanoparticles)

A fluorescent labeling agent related to the present invention is characterized in containing activated fluorescent nanoparticles which comprises (1) a fluorescent labeling agent core part which comprises at least three activated fluorescent nanoparticles per a fluorescent labeling agent and a protective material; and (2) an organic surface-coating layer which coats the fluorescent labeling agent core part, wherein the fluorescent labeling agent core part has an average particle diameter of 10 to 50 nm.

As an activated fluorescent nanoparticles related to the present invention, various types of activated fluorescent nanoparticles containing activator can be employed. As the activated fluorescent nanoparticles, either a host excitation type fluorescent nanoparticle, for example, ZnSiO₄ in which host excited energy is absorbed by an activator to result in light emission or activator excited type fluorescent nanoparticle in which activator itself is excited to result in light emission. According to the present invention, the latter is preferable.

According to the present invention, near-infrared emitting fluorescent nanoparticle described later is specifically preferable.

(Near-Infrared Emitting Fluorescent Nanoparticles)

Near-infrared emitting fluorescent nanoparticles related to the present invention has an average particle size of 2-50 nm and emits near-infrared light having a wavelength in the range of 700-2,000 nm when excited by a near-infrared light having a wavelength in the range of 700-900 nm. At least a part of its composition is represented by Formula (1): APO₄, or Foiinula (2): AF₃ (wherein, A is an atom selected from yttrium (Y), lutetium (Lu) and lanthanum (La), and preferably comprises a rare earth element as an activator.

The rare earth element is preferably any one or combination of praseodymium (Pr), neodymium (Nd), holmium (Ho), erbium (Er) or ytterbium (Yb).

Further, as a co-activator, at least one element of Pr or Tb is preferably employed. Herein, “co-activator” means the second component which helps a function of activator.

When finally fanned near-infrared emitting fluorescent nanoparticles are particles of 50 nm or less, and further when number of type of metal element in constituting element are four or more or less than 10 atom % of co-activator is included, extremely high luminous intensity can be obtained, comparing to particles prepared by conventional solid phase method or particles having three types of metal elements or without having co-activator.

As a method for producing near-infrared emitting fluorescent nanoparticles, for example, method described in Nano Letters Vol. 2, 733-737 (2002) or Chemistry of Materials Vol. 15, 4604-4616 (2003) can be applicable.

As raw material for producing near-infrared emitting fluorescent nanoparticles, employed are halide or nitrate salt of various elements contained in Formula (1) or Formula (2). For example, neodymium chloride, neodymium nitrate, ytterbium chloride, ytterbium nitrate, lanthanum chloride, lanthanum nitrate, yttrium chloride, yttrium nitrate, praseodymium chloride and erbium chloride are usable.

As phosphoric acid source, ortho phosphoric acid and as fluoride source, sodium fluoride can be usable.

(Organic Surface-Coating Layer)

A fluorescent labeling agent related to the present invention is characterized in containing quantum dots which comprises (1) a core part which comprises at least three quantum dots per a fluorescent labeling agent and a protective material; and (2) an organic surface-coating layer which coats the core part.

As a method for forming the organic surface-coating layer, various methods conventionally known in the art can be employed. As a material for comprising the organic surface-coating layer, various materials can be employed.

Example of general method for forming the organic surface-coating layer and a material for comprising the organic surface-coating layer is as below: surface of quantum dots (semiconductor nanoparticles) is hydroxylated by using hydrogen peroxide solution, followed by reacting silane coupling agent having functional group such as mercapto group or/and amino group onto hydroxylated surface. Then, compound (polyethylene glycols) having polyethylene glycol chain which has reactive functional group with the functional group is reacted to prepare an organic surface-coating layer of embodiment related to the present invention.

As silane coupling agent related to the present invention, silane compound represent by Formula below or derivatives thereof can be employable.

Formula: X-A-Si(OR)ₙR′ₙ₋₃, wherein n=3, X represents a functional group such as amino group, mercapto group, halogen, epoxy group, vinyl group, methacryloxy group, acryloxy group, and N-(aminoalkyl)-amino group.

A represents hydrocarbon chain such as —(CH₂)₂—, —(CH₂)₃— and —(CH₂)₄—; R and R′ represents straight chain or branched chain, generally having carbon number of 1-6, and may be the same or different.

Specific example of these silane coupling agent include: N-(2-aminoethyl)-3-aminopropylmethyldimethoxysilane, N-(2-aminoethyl)-3-aminopropyltrimethoxysilane, N-(2-aminoethyl)-3-aminopropyltriethoxysilane, 3-aminopropyltrimethoxysilane, 3-aminopropyltriethoxysilane, 3-triethoxysilyl-N-(1,3-dimethyl-butylidene) propylamine, N-phenyl-3-aminopropyltrimethoxysilane, N-(vinylbenzyl)-2-aminoethyl-3-aminopropyltrimethoxysilane, 3-ureido propyltriethoxysilane, 3-chloropropyltrimethoxysilane, 3-mercaptopropylmethyldimethoxysilane, 3-mercaptopropyltrimethoxysilane, bis(triethoxysilylpropyl)tetrasufide, 3-isocianatepropyltriehoxysilane, 2-(3,4-epoxycyclohexyl)ethyltrimethoxysilane, 3-glycidoxypropyltrimethoxysilane, 3-glycidoxypropylmethyldimethoxysilane, 3-glycidoxypropyltriethoxysilane, 3-methacryloxypropylmethyldimethoxysilane, 3-methacryloxypropyltrimethoxysilane, 3-methacryloxypropylmethyldiethoxysilane, 3-methacryloxypropyltriethoxysilane, 3-acryloxypropyltrimethoxysilane, p-stylyltrimethoxysilane, vinyltrichlorosilane, vinyltrimethoxysilane, vinyltriethoxysilane, vinyltriacetoxysilane, and octadecyldimethyl[3-(trimethoxysilyl)propyl]ammonium chloride. Among them, silane compound having amino group or mercapto group is preferably employed to the present invention.

Coupling agent may be used in diluted solution using dispersing solvent below. Generally, it is used as aqueous solution, but in some cases, may be used in a form of aqueous solution in which a small amount of acetic acid is added. Concentration of coupling agent may be used in appropriate. For example, coupling agent concentration of 0.001-5.0% or 0.01-1.0% may be added to a dispersed liquid of quantum dots (semiconductor nanoparticles).

While dispersing solvent usable in the present invention can not be defined indiscriminately, because solubility of surface modifier is different by species, listed are water, ketones such as acetone or methylethylketone; esters such as ethylacetate; alcohols such as methanol or ethanol; non-protonic polar solvents such as dimethylformamide, dimethylsulfoxide, sulforan, digrim, or hexamethyltriamidephosphate; nitromethane and acetonitril. Specifically, hydrophilic organic solvent such as water, alcohol or ketone mixed with water may be preferably employed.

According to the present invention, various types of organic polymer materials can be employed as a material for forming the organic surface-coating layer.

As organic polymer materials, polymer obtained by polymerization of polymerizable monomer below can be employable.

Specific example of polymerizable monomer include styrene or styrene derivative such as styrene, o-methylstyrene, m-methylstyrene, p-methylstyrene, α-methylstyrene, p-chlorostyrene, 3,4-dichlorostyrene, p-phenylstyrene, p-ethylstyrene, 2,4-dimethylstyrene, p-tert-butylstyrene, p-n-hexylstyrene, p-n-octylstyrene, p-n-nonylstyrene, p-n-decylstyrene, and p-n-dodecylstyrene.

Further, polymerizable monomer include methacrylic ester derivatives such as methyl methacrylate, ethyl methacrylate, n-butyl methacrylate, isopropyl methacrylate, isobutyl methacrylate, t-butyl methacrylate, n-octyl methacrylate, 2-ethylhexyl methacrylate, stearyl methacrylate, lauryl methacrylate, phenyl methacrylate, diethylaminoethyl methacrylate, and dimethylaminoethyl methacrylate; and acrylic ester derivatives such as methyl acrylate, ethyl acrylate, isopropyl acrylate, n-butyl acrylate, t-butylacrylate, isobutyl acrylate, n-octyl acrylate, 2-ethylhexyl acrylate, stearyl acrylate, lauryl acrylate, and phenyl acrylate.

Further, polymerizable monomer include olefin such as ethylene, propylene or isobutylene; halogenated vinyls such as vinyl chloride, vinylidene chloride, vinyl bromide, vinyl fluoride, or vinylidene fluoride; vinyl esters such as vinyl propionate, vinyl acetate or vinyl benzoate; vinyl ethers such as vinyl methylether or vinyl ethylether; vinyl ketones such as vinyl methyl ketone, vinyl ethyl ketone, or vinyl hexyl ketone; N-vinyl compound such as N-vinyl carbazole, N-vinyl indole or N-vinyl pyrolidone; vinyl compound such as vinyl naphthalene or vinyl pyridine; and acryl acid or methacryl acid derivatives such as acrylonitrile, methacrylonitrile or acryamide.

These vinyl based monomer may be employed individually or in combinations.

Further, more preferred is to combine with compound having ionic dissociable group as polymerizable monomer constituting an organic polymer. For example, substituent such as carboxyl group, sulfonic acid group, and phosphoric acid group is listed as a constituting group of monomer. Specific example include acrylic acid, methacrylic acid, maleic acid, itaconic acid, cinnamic acid, fumaric acid, monoalkyl maleate ester, monoalkyl itaconate ester, styrene sulfonic acid, arylsulfo succinic acid, 2-acrylamide-2-methylpropane sulfonic acid, acidphosphoxy ethylmethacrylate, and 3-chloro-2-acidphosphoxy propylmethacrylate.

Further, polyfunctional vinyls such as divinyl benzene, ethyleneglycol dimethacrylate, ethyleneglycol diacrylate, diethyleneglycol dimethacrylate, diethyleneglycol diacrylate, triethyleneglycol dimethacrylate, triaethyleneglycol diacrylate, neopentylglycol dimethacrylate, or neopentylglycol diacrylate may be employable to form resin having cross-linked structure.

According to the present invention, specifically preferred material is copolymer synthesized by monomer composition such as styrene, n-butylacrylate and methacrylic acid. Specific example includes a copolymer synthesized by monomer composition of: styrene (107 parts by mass), n-butylacrylate (50 parts by mass), methacrylic acid (8 parts by mass) and octylmercaptane (chain transfer agent: 4 parts by mass).

(Chemically-Modification of Organic Surface-Coating Layer)

The organic surface-coating layer of the present invention is preferably chemically-modified by a compound represented by Formula (PEG):

X—(OCH₂CH₂O)ₙ—Y, Formula (PEG)

wherein X represents a linkage group containing a linking group at terminal to a compound constituting the organic surface-coating layer, Y represents hydroxyl group, alkoxy group, and a functional group at terminal bondable to a biomolecule, and n represents an integer of 1-20.

Herein, “a group at terminal containing a functional group bondable to a biomolecule” is referred to as a group at terminal of chemical structure such as carboxyl group, amio group, phosphonic acid group, sulfonic acid group, or mercapto group which can bond to a biomolecule as examples below.

Compound having polyethyleneglycol chain (polyethylenglycols) usable to the present invention may not be limited, as long as it has polyethyleneglycol chain. Specific example include polyethyleneglycols such as HS—C₂H₄(OCH₂CH₂)ₙ—OCH₃, NH₂—C₂H₄(OCH₂CH₂)ₙ—OCH₃, C(═O)H—C₂H₄(OCH₂CH₂)ₙ—OCH₃, NH₂—C₂H₄—(OCH₂CH₂)ₙ—OC(═O)O-succinimide, maleimide-(CH₂)₂C(═O)NHC₃H₆—(CH₂CH₂O)ₙ—OC(═O)O-succinimide, HO—(CH₂CH₂O)ₙ—CH₂CH₂C(═O)H, HO—(CH₂CH₂O)ₙ—C₃H₆NH₂, H₂N(CH₂)₃₀(CH₂CH₂)ₙ(CH₂)₅C(═O)OH, and biotin-(CH₂)₄C(═O)₂NH(CH₂CH₂O)ₙC(═O)O-succinimide. In above compound, n represents an integer of 1 to 20, and preferably 2 to 10.

Following method is preferable to apply as a method for chemically-modification.

Chemically-modification is carried out by dehydration reaction via stirring under a room temperature, resulting in obtaining amide bonding. Catalyst may be used.

(Fluorescent Labeling Agent)

The fluorescent labeling agent of the present invention is adaptable to a fluorescent labeling agent which can fluorescent label a target substance by arranging adequate surface modifying compound on quantum dots (semiconductor nanoparticles).

Specifically, the fluorescent labeling agent is adaptable to a biomolecule fluorescent labeling agent (a biological substance fluorescent labeling agent) for detecting a target substance such as protein or peptide by arranging adequate surface modifying compound having affinity or conjugating to living body on the particles.

In the case of a biological molecule fluorescent labeling agent (a biological substance fluorescent labeling agent), in view of noninvasive to a biomolecule and transparency to living tissue, preferred is to arrange emission property of semiconductor nanoparticles by controlling particle size to emit infrared ray by excitation of near-infrared to infrared.

According to the present invention, surface modifying compound is preferred to be a compound having at least one functional group and at least one group bonding to semiconductor nanoparticle. The latter group is a group which can be absorbed to hydrophobic semiconductor nanoparticle and the former group has hydrophilic and can be bonded to a biological substance. Various types of linkers can be usable to link each surface modifying compound.

For example, a group bonding to semiconductor nanoparticle may be a functional group which bonds to semiconductor material to form the semiconductor nanoparticles. In the present invention, mercapto group (thiol group) is specifically preferred as the functional group.

As a group bonding to a biological substance with affinity, listed are carboxyl group, amino group, phosphonic acid group and sulfonic acid group.

Herein, “biological substance” is referred to cell, DNA, RNA, oligonucleotide, protein, antigen, antibody, endoplasmic reticulum, nucleus and Golgi body.

Mercapto group can be bonded to semiconductor nanoparticle by adjusting pH suitable for surface-modification. Aldehyde group, amino group or carboxyl group can be introduced to the other end which can result in peptide bonding to amino group or carboxyl group in a living body. The same bonding can be obtained by introducing amino group, aldehyde group or carboxyl group in DNA and oligonucleotide.

As specific example of a method for preparing a biomolecule fluorescent labeling agent (a biological substance fluorescent labeling agent) by using semiconductor nanoparticle related to the present invention, listed is a method in which hydrophilized semiconductor nanoparticle is bonded to molecule labeling substance through organic molecule. In the biomolecule fluorescent labeling agent (the biological substance fluorescent labeling agent) prepared by this method, molecule labeling substance is peculiarly bonded and/or reacted to targeted biological substance and results in fluorescent labeling.

Molecule labeling material includes nucleotide chain, antibody, antigen, and cyclodextrin.

Organic molecule is not limited unless it can bond semiconductor nanoparticle with molecule labeling material. For example, preferably employed is protein such as albumin, myoglobin and casein; combination of avidin that is a kind of protein with biotin. Type of bonding is not limited and employed are covalent bond, ionic bond, hydrogen bond, coordinate valence bond, physical absorption and chemical absorption. In view of stability of bonding, preferred is a bond with strong bonding such as covalent bond.

In specific example in which semiconductor nanoparticles are hydrophilized by mercaptodecane acid, avidin and biotin are employable as organic molecule. In this case, carboxy group in the hydrophilized nanoparticles bonds to avidin by covalent bond, avidin further electively bonds to biotin, biotin further bonds to molecule labeling material and results in a biomolecule fluorescent labeling agent (a biological substance fluorescent labeling agent).

[Hydrophilization of Semiconductor Nanoparticle]

The surface of the above semiconductor nanoparticle is usually hydrophobic. Therefore, for example, when the semiconductor nanoparticle is used as a biomolecule labeling agent as it is, it exhibits poor water dispersibility, resulting in agglomerated particles which are problematic, whereby the surface of a semiconductor nanoparticle is preferably hydrophilized.

A method of hydrophilization includes, for example, a method wherein lipophilic groups on the surface are removed by pyridine and then a surface modifier is allowed to bond to the particle surface in at least one manner of a chemical and a physical manner. As the surface modifier, those containing a carboxyl group or an amino group as a hydrophilic group are preferably used. There are specifically listed, for example, mercaptopropionic acid, mercaptoundecanoic acid, and aminopropanethiol. Specifically, for example, 10⁻⁵ g of a Ge/GeO₂ type nanoparticle is dispersed in 10 ml of purified water dissolving 0.2 g of mercaptoundecanoic acid and stirred at 40° C. for 10 minutes, followed by surface treatment of the shell to modify the shell surface of the inorganic nanoparticle with a carboxyl group.

Specific preparation method for surface modification of seminconductor nanoparticles can be carried out based on the method described in Dabbousi et al, (1997) J. Phys. Chem. B101: 9463, Hines et al, (1996) J. Phys. Chem. 100: 468-471, Peng et al, (1997) J. Am. Chem. Soc. 119: 7019-7029 and Kuno et al, (1997) J. Phys. Chem. 106: 9869.

(Fluorescent Labeling Agent and Biomolecule Detection System Using Thereof)

The fluorescent labeling agent of the present invention having above characteristics is preferably adaptable to a biomolecule detection system characterized in which the fluorescent labeling agent is applied to the targeted living cell or living tissue, emitted fluorescence from semiconductor nanoparticles by radiation excitation is detected and the biomolecule in the targeted living cell or living tissue can be detected.

When added to a living cell or a living tissue having a target (trace) biomolecule, a fluorescent labeling agent according to the present invention is bonded or adsorbed to the target molecule. Thereafter, the resulting bonded or adsorbed body is irradiated by excitation light (radiation) having a predetermined wavelength and then fluorescence of a specific wavelength emitted from a semiconductor nanoparticle (a fluorescent semiconductor fine particle) according to the excitation light is detected. Thereby, fluorescent dynamic imaging of the target (trace) biomolecule can be carried out. Namely, the fluorescent labeling agent of the present invention can be utilized in a bioimaging method (a technological method to visualize biomolecule constituting a biological substance and dynamic phenomena thereof).

Radiation for excitation includes visible light such as halogen lamp and tungsten lamp, LD, near-infrared laser light, infrared laser light, X-ray and γ ray.

(Molecule/Cell Imaging Method)

A quantum dot according to the present invention can be used as a fluorescent labeling agent by bonding a probe molecule (a detecting molecule) which electively reacts with molecule existing inner or on a surface of targeted cell tissue.

Herein, “target” means a biomolecule targeted by a semiconductor nanoparticle, for example, protein preferentially expressed in tissue and cell, Golgi body in cell, nucleus and membrane protein. Examples of preferred target substance include enzyme and protein, cell surface receptor, nucleic acid; lipid and phospholipid, without being restricted thereto.

According to the present invention, preferred is to apply adequate probe molecule corresponding to targeted (measured) substance, for the purpose of visualizing inside of living body or dynamic measurement of the substance in a cell.

The fluorescent labeling agent (a biomolecule fluorescent labeling agent) utilizing semiconductor nanoparticles can be applied to various types of molecule/cell imaging method conventionally known in the art. Specific examples include molecule/cell imaging method such as laser injection method, micro injection method, and electroporation method. Of these, laser injection method is preferably applied to molecule/cell imaging method.

Herein, “laser injection method” is an optical method in which laser is irradiated directly to a cell and then foreign substance such as gene is introduced by directly injection through fine hole opened at cell.

“Micro injection method” is a mechanical method in which foreign substance such as gene is introduced into cell by directly injection by using fine needle (micro pipet, micro syringe) via air pressure.

“Electroporation method” is a physical method in which foreign substance such as gene is introduced into cell by applying electric stimulation to a cell to induce distortion of cell. For example, when high voltage as several thousands V/cm is applied to cell suspension liquid in pulse of several ten micro seconds, tiny hole is generated at cell membrane in a short time and through this tiny hole, and outer liquid may be took into cell. Therefore, when sample such as DNA which is intended to inject into is added in outer liquid of cell, it can be introduced into cell. Electroporation method makes use of above process.

### EXAMPLES

Embodiments of the present invention will now be specifically described with the reference to examples, however the present invention is not limited thereto.

### Example 1

### Preparation of Quantum Dot

(Preparation of Si/SiO₂ Core/Shell Particles)

(HF Etching Method)

In a preparation of a semiconductor nanoparticle of Si (hereinafter, refers to as “Si semiconductor fine particle” or “Si core particle”) by dissolving heat treated SiOₓ (×1.999) in fluoric acid, at first, SiO, (x≦1.999) foamed on silicone wafer by plasma CVD was annealed under atomosphere of inert gas at 1,100° C. Thereby, Si semiconductor fine particle (crystalline) was separating. By controlling annealing time, Si fine particles having different size were separating.

Consequently, silicon wafer was treated to eliminate SiO₂ layer by about 1% of fluoric acid aqueous solution at room temperature and Si semiconductor fine particles of several nm size were collected which were coagulated at surface of liquid. This fluoric acid treatment lead to terminate dangling bond (un-bonding hand) of Si atom at the surface of semiconductor fine particles by hydrogen-termination to result in stabilize Si crystalline. Then, surface of collected Si semiconductor fine particles was heat oxidized under oxygen circumstances by heating about 1.5 hours at 800-1,000° C. to form shell layer of SiO₂ around core of Si semiconductor fine particles.

(Preparation of Si/ZnS Core/Shell Particles)

Si core particles obtained above were dispersed in pyridine and kept at 100° C. Separately, Zn(C₂H₅)₂, ((CH₃)₃Si)₂S and P(C₄H₉)₃ were slowly mixed under argon gas circumstance with applying ultrasonic wave at 100° C.

Mixture was dropped to add into pyridine dispersion. After addition, temperature was kept at 100° C., pH (8.0) was kept at constant and slowly stirred in 30 minutes. Centrifugal separation was carried out and settled particles were collected. Si and ZnS were confirmed by element analysis of obtained particles. By XPS analysis, it was found that ZnS covered a surface of Si.

Average particle size of above core/shell semiconductor nanoparticles of Si/SiO₂ and Si/ZnS were measured by using Zetasizer manufactured by Sysmex Corp. The results were listed in Table 1.

(Preparation of Fluorescent Labeling Agent Core Part)

Cyclohexane and polyoxyethylene nonylphenylether were mixed and quantum dots having species and particle size listed in Table 1 were dispersed in water. This liquid was added by stirring vigorously so as to water/surfactant ratio being 2.0, to form a reverse micelle. Tetraethoxysilane (TEOS) was added to be 1/100 based on water, and quantum dots were coated. By adding one drop of NH₄OH aqueous solution as catalyst, hydrolysis of TEOS was completed under alkali pH region. Further, mixture solution was sealed and stirred 24 hours to accelerate coating and after sedimentation, core part of fluorescent labeling agent was obtained. Average particle size was measured by using Zetasizer manufactured by Sysmex Corp. The results were listed in Table 1.

Numbers of quantum dots in core part of fluorescent labeling agent were calculated by lattice image of quantum dots in photographs by TEM (transmission electron microscope).

As shown in Table 1, numbers of quantum dots in core part of fluorescent labeling agent were controlled by arranging a concentration of quantum dot dispersion aqueous solution.

(Introduction of Surface-Modifying Compound)

Into above solution, 3-aminopropyltriethoxysilane (APS) ethanol solution was added and stirred by arranging pH at about 6.5 to graft APS to surface of core part of fluorescent labeling agent. Then, miscelle was destroyed by adding 0.02M acetic acid/ethanol solution with stirring vigorously. This suspension was purified by using HPLC (Delta Preparation 3000 HPLC system, manufactured by Waters) with HR5/5 column (manufactured by Amersham Pharmacia Biotech, Inc.) filled with 20 μm of spherical silica beads treated by APS. End of the column was connected to a UV-visible spectrum detector which can detect peak wavelength of fluorescence from quantum dots. Absolute ethanol was pump fed to HPLC as washing solution and APS introduced nano labeling agent was recovered by solution of ethanol/water=7/3. By using fraction collector, elute from HPLC column in all washing process were gathered. To this gathered elute, polyethylene glycol compound having succinimide group (NHS) and methoxy group at its end manufactured by Nippon Yushi Industry Co., Ltd. (molecular weight: described in Table 1) was added and stirred 24 hours at room temperature. Again, by using size-exclusion chromatography method (GPC apparatus), un-reacted polyethylene glycol was separated and elute from GPC column was collected by using fraction collector. Ethanol in this elute was replaced to water by adding water while eliminating by vacuum condition.

(Example of Observation of Fluorescent Labeling Biomolecule)

Collected labeling agent aqueous solution above was preliminary mixed with sheep serum albumin (SSA) in same concentration, and taken into Vero cell individually. After cultivating 24 hours at 37° C., it was treated with trypsin, followed by suspending in DMEM with 5% FBS again, and seeded on the same glass bottom dish. Cell was cultivated one night at 37° C., fixed by 4% formalin, dyed its nucleus by DAPI, and fluorescence was observed by a cofocal laser scan micrometer (excited at 405 nm).

Accumulated state of the labeling agent taken into endosome of cytoplasm was evaluated by density and dispersion state which were found from luminous intensity. Namely, when transfer efficiency of labeling agent after taken into cell and transfered to and accumulated at endosome is uniform and high, fluorescence strength at endosome is high and its distribution is also uniform and spreads over a large area. This represents that there is no coagulation and bonding on labeling agent itself and also no nonspecific absorption occurs. On the other hand, when efficiency of taken into or mobility is low due to coagulation and nonspecific absorption, fluorescence strength is low and uneven mottles such that emission extremely differs its strength at its position and accumulated emission area is small. The results are listed in Table 1.

(Stability Test of Labeling Agent for Environmental Condition)

In case that pH value, concentration of NaCl and temperature are changed and stored, change of luminous intensity property of separately collected labeling agent aqueous solution above is traced. The results are listed in Table 2.

TABLE 1

| | Quantum dot | | | | | | | | |
|---|---|---|---|---|---|---|---|---|---|
| | Ave. | | Fluorescent labeling | | | Molecular | Ave. | | |
| | particle | | agent core part | | | weight of | luminous | | |
| Sample | size | | No. of quantum | Particle size | Protective | Formula | intensity | | Remarks |
| No. | (nm) | Species | dots | (min) | Material | (PEG) | (*) | Observation of Cell imaging | ** |
| 1 | 3.2 | Si/SiO₂ | 3 | 22 | SiO₂ | 1000 | 100 | Uniform emission and shape of cell | Inv. |
| | | | | | | | | membrane was clearly noted. | |
| 2 | 3.2 | Si/SiO₂ | 5 | 22 | SiO₂ | 1000 | 130 | Uniform emission and shape of cell | Inv. |
| | | | | | | | | membrane was clearly noted. | |
| 3 | 3.2 | Si/SiO₂ | 9 | 22 | SiO₂ | 1000 | 180 | Uniform emission and shape of cell | Inv. |
| | | | | | | | | membrane was extremely clearly noted. | |
| 4 | 3.2 | Si/SiO₂ | 15 | 22 | SiO₂ | 1000 | 140 | Uniform emission and shape of cell | Inv. |
| | | | | | | | | membrane was clearly noted. | |
| 5 | 3.2 | Si/SiO₂ | 1 | 22 | SiO₂ | 1000 | 50 | Emission was poor, uneven and blurred. | Comp. |
| 6 | 3.2 | Si/SiO₂ | 2 | 8 | SiO₂ | 1000 | 70 | Emission is poor, uneven and blurred | Comp. |
| 7 | 3.2 | Si/SiO₂ | 5 | 70 | SiO₂ | 1000 | 110 | Introduction to cell was extremely low and | Comp. |
| | | | | | | | | emission was blurred. | |
| 8 | 3.2 | Si/SiO₂ | 5 | 22 | SiO₂ | Free | 175 | Bright but slightly coagulated and slightly | Inv. |
| | | | | | | | | uneven emission. | |
| 9 | 3.2 | Si/ZnS | 5 | 24 | ZnS | 1000 | 140 | Uniform emission and shape of cell | Inv. |
| | | | | | | | | membrane was clearly noted. | |
| 10 | 3.2 | Si/ZnS | 9 | 24 | ZnS | 1000 | 200 | Uniform emission and shape of cell | Inv. |
| | | | | | | | | membrane was clearly noted. | |
| 11 | 3.2 | Si/ZnS | 2 | 8 | ZnS | 1000 | 65 | Emission was poor, uneven and blurred. | Comp. |
| 12 | 3.2 | Si/ZnS | 5 | 70 | ZnS | 1000 | 115 | Introduction to cell was extremely low and | Comp. |
| | | | | | | | | emission was blurred | |

*Represented based on Sample No. 1 as 100.
**Inv.: Inventive example, Comp.: Comparative example

TABLE 2

| | | | | Conc. of | Luminous intensity | | | |
|---|---|---|---|---|---|---|---|---|
| Eval. | Sample No. | | Temp. | NaCl | Immediately | 3 days | 1 month | Remarks |
| No. | (Same as Table 1) | pH | (° C.) | (mol/l) | (*) | after | after | ** |
| 1 | 3 | 7 | 25 | 0.05 | 100 | 100 | 98 | Inv. |
| 2 | 3 | 5 | 25 | 0.05 | 98 | 100 | 97 | Inv. |
| 3 | 3 | 8.5 | 25 | 0.05 | 98 | 99 | 98 | Inv. |
| 4 | 3 | 7 | 40 | 0.05 | 100 | 100 | 98 | Inv. |
| 5 | 3 | 7 | 25 | 0.5 | 100 | 100 | 97 | Inv. |
| 6 | 3 | 7 | 40 | 0.5 | 100 | 100 | 96 | Inv. |
| 7 | 6 | 5 | 25 | 0.05 | 70 | 65 | 50 | Comp. |
| 8 | 6 | 7 | 40 | 0.05 | 65 | 60 | 45 | Comp. |
| 9 | 6 | 7 | 25 | 0.5 | 60 | 55 | 30 | Comp. |
| 10 | 6 | 7 | 40 | 0.5 | 55 | 40 | 10 | Comp. |

*Represented based on Sample No. 1 as 100.
“Luminous intensity at immediately” means a luminous intensity immediately after preparing aqueous solution containing labeling agent (within 15 minutes).
**Inv.: Inventive example, Comp.: Comparative example

As described in Table 1, it is found that the fluorescence labeling compound related to the present invention does not coagulate, exhibits excellent dispersability and detectability of labeling for targeted living body is extremely stable and clear. Further, as shown in Table 2, it is found that high stability can be established without depending to environmental condition by using constitution of the present invention. Namely, the present invention can provide a fluorescent labeling agent containing quantum dots, which exhibits little fluctuation in luminous properties in accordance with the environment and therefore has high environmental stability, and which has high luminous intensity.

### Example 2

### Preparation of Activated Fluorescent Nanoparticles

### LaPO₄: Nd (Fluorescence 1)

By using lanthanum chloride and neodymium chloride, LaPO₄: Nd nanoparticles were synthesized by the method described in Chemistry of Materials Vol. 15, 4604-4616 (2003).

Average particle size of LaPO₄: Nd measured by using Zetasizer manufactured by Sysmex Corp was 4 nm.

(Preparation of Fluorescent Labeling Agent Core Part)

Cyclohexane and polyoxyethylene nonylphenylether were mixed and liquid in which LaPO₄: Nd was dispersed in water was added by stirring vigorously so as to water/surfactant ratio being 2.0, to form a reverse micelle. Tetraethoxysilane (TEOS) containing 1/200 of neodymium chloride based on fluorescence was added to be 1/100 based on water, and coated by LaPO₄: Nd. By adding one drop of NH₄OH aqueous solution as catalyst, hydrolysis of TEOS was completed under alkali pH region. Further, mixture solution was sealed and stirred 24 hours to accelerate coating and after sedimentation, core part of fluorescent labeling agent was obtained. Average particle size was measured by using Zetasizer manufactured by Sysmex Corp. The results were listed in Table 3.

Numbers of LaPO₄: Nd in core part of fluorescent labeling agent were calculated by lattice image of quantum dots in photographs by TEM (transmission electron microscope).

As shown in Table 3, numbers of LaPO₄: Nd in core part of fluorescent labeling agent were controlled by arranging a concentration of quantum dot dispersion aqueous solution.

(Introduction of Surface-Modifying Compound)

Into above solution, 3-aminopropyltriethoxysilane (APS) ethanol solution was added and stirred by arranging pH at about 6.5 to graft APS to surface of core part of fluorescent labeling agent. Then, miscelle was destroyed by adding 0.02M acetic acid/Ethanol solution with stirring vigorously. This suspension was purified by using HPLC (Delta Preparation 3000 HPLC system, manufactured by Waters) with HR5/5 column (manufactured by Amersham Pharmacia Biotech, Inc.) filled with 20 μm of spherical silica beads treated by APS. End of the column was connected to a UV-visible spectrum detector which can detect peak wavelength of fluorescence from quantum dots. Absolute ethanol was pump fed to HPLC as washing solution and APS introduced nano labeling agent was recovered by solution of ethanol/water=7/3. By using fraction collector, elute from HPLC column were gathered in all washing process. To this gathered elute, polyethylene glycol compound having succinimide group (NHS) and methoxy group at its end manufactured by Nippon Yushi Industry Co., Ltd. (molecular weight: described in Table 1) was added and stirred 24 hours at room temperature. Again, by using size-exclusion chromatography method (GPC apparatus), un-reacted polyethylene glycol was separated and elute from GPC column was collected by using fraction collector. Ethanol in this elute was replaced to water by adding water while eliminating by vacuum condition.

Further, four kind of protein or antibody which were specific absorbed on biomarker which appeared specifically at each stage of breast cancer were absorbed on the labeling agent comprising Si/SiO₂ core/shell particle having different size listed in Table 1.

### Preparation of Comparative Example

Comparative example A: Si/SiO₂ was prepared by controlling particle size so as to have emitting wavelength at 550 nm and coated, introduced surface modifying compound as the same manner. Further, as the same manner above, protein or antibody which was specific bonded to biomarker was absorbed.

Comparative example B: Near-infrared dye was used as labeling agent. Terminal structure of dye by Vizen was modified so as to labeling (targeting) to breast cancer tissue and target protein (protein to bond to target) was absorbed.

<Method for Disease Examination>

Into vein of five patients of breast cancer whose stage of cancer were unknown, the fluorescent labeling agent of the present invention was injected. Laser of excitation wavelength of 650 nm was irradiated against the breast and emission was caught by cooling type high sensitivity CCD camera to imaging observation.

Further, in the same manner, comparative example 1 was injected to patients of breast cancer. Excitation light of 400 nm and 650 nm were irradiated and imaging was observed. Comparative example 2, near-infrared dye, was irradiated by Excitation light of 650 nm and imaging was observed.

The results of observation were listed in Table 3.

TABLE 3

| | Fluorescent labeling agent | | | | | | | |
|---|---|---|---|---|---|---|---|---|
| | core part | | | | | | | |
| | Activated fluorescent | | | | Wavelength | | | |
| | nanoparticle | | Particle | | of excitation | Wavelength of | | |
| Sample | | No. of | size | Protective | light | emission | Observation of Cell imaging | Remarks |
| No. | Species | particle | (nm) | Material | (nm) | (nm) | Determination of Stage of Breast cancer | * |
| 1 | LaPO₄:Nd | 3 | 24 | SiO₂ | 650 | 725 | Emission was detected. Affected part was clearly | Inv. |
| | | | | | | | confirmed. Stage was determined. | |
| 2 | LaPO₄:Nd | 5 | 24 | SiO₂ | 650 | 725 | Emission was detected. Affected part was clearly | Inv. |
| | | | | | | | confirmed. Stage was determined. | |
| 3 | LaPO₄:Nd | 9 | 24 | SiO₂ | 650 | 725 | Emission was detected. Affected part was clearly | Inv. |
| | | | | | | | confiimed. Stage was determined. | |
| 4 | LaPO₄:Nd | 15 | 24 | SiO₂ | 650 | 725 | Emission was slightly weak but detected. Affected | Inv. |
| | | | | | | | part was confirmed. | |
| 5 | LaPO₄:Nd | 5 | 16 | SiO₂ | 650 | 725 | Emission was detected. Affected part was clearly | Inv. |
| | | | | | | | confirmed. Stage was determined. | |
| 6 | LaPO₄:Nd | 9 | 37 | SiO₂ | 650 | 725 | Emission was detected. Affected part was clearly | Inv. |
| | | | | | | | confirmed. Stage was determined. | |
| 7 | LaPO₄:Nd | 3 | 7 | SiO₂ | 650 | 725 | Emission was weak and affected part was blurred. | Comp. |
| 8 | LaPO₄:Nd | 2 | 70 | SiO₂ | 650 | 725 | Labeling agent may not reach to affected part. No | Comp. |
| | | | | | | | emission was noted | |
| 9 | LaPO₄:Nd | 3 | 70 | SiO₂ | 650 | 725 | Emission was weak and affected part was extremely | Comp. |
| | | | | | | | blurred. | |
| 10 | LaPO₄:Nd | 1 | 24 | SiO₂ | 650 | 725 | Emission was weak and blurred. | Comp. |
| 11 | Si/SiO₂ | — | — | — | 400 | 550 | Emission was weak. | Comp. A |
| 12 | Si/SiO₂ | — | — | — | 650 | — | No emission was noted. | Comp. A |
| 13 | Near-infrared | — | — | — | 700 | 720 | Emission was extremely weak. | Comp. B |
| | dye | | | | | | | |

*Inv.: Inventive example, Comp.: Comparative example

As can seen from Table 3, as compared that the fluorescent labeling agent according to the present invention can detect the affected part, while comparative example A cannot detect at all. It is because that emission of comparative example A is in visible light region and is absorbed into a living body. On the other hand, the optical detective labeling agent of the present invention containing activated fluorescent nanoparticles emit near-infrared emission and thereby transmitted light through living tissue can be detected. As for comparative example B, emission can be detected but very weak and accuracy of detection is extremely poor than the fluorescent labeling agent according to the present invention.

Further, as can seen from the difference between comparative examples 1 to 3 in Table 3, the constitution of the present invention is superior to detectability and effective to diagnosis as the optical detective labeling agent.

1. A fluorescence labeling agent containing quantum dots comprising:
(1) a fluorescence labeling agent core part which comprises at least three quantum dots per a fluorescence labeling agent and a protective material; and
(2) an organic surface-coating layer which coats the fluorescence labeling agent core part,
wherein the fluorescence labeling agent core part has an average particle diameter of 10 to 50 nm.

2. The fluorescence labeling agent of claim 1, wherein the quantum dots comprising silicon (Si).

3. The fluorescence labeling agent of claim 1, wherein
the quantum dots comprises activated fluorescent material nanoparticles, near-infrared fluorescent material nanoparticles emitting near-infrared light having a wavelength in the range of 700-2,000 nm,
at least a part of composition of the quantum dots is represented by Formula (1): APO₄, or Formula (2): AF₃, wherein A is an atom selected from yttrium (Y), lutetium (Lu) and lanthanum (La), and comprises a rare earth element as an activator.

4. The fluorescence labeling agent of claim 3, wherein the rare earth element is any one or combination of praseodymium (Pr), neodymium (Nd), holmium (Ho), erbium (Er) or ytterbium (Yb).

5. The fluorescence labeling agent of claim 1, wherein the protective material is SiOₓ, wherein x=1.5-2.0, or ZnS.

6. The fluorescence labeling agent of claim 1, wherein a number of the quantum dots per one fluorescence labeling agent is 3-10.

7. The fluorescence labeling agent of claim 1, wherein the organic surface-coating layer is chemically-modified by a compound represented by Formula (PEG),
X—(CH₂CH₂O)ₙ—Y, Formula (PEG)
wherein X represents a linkage group containing a linking group at terminal linkable to a compound constituting the organic surface-coating layer, represents hydroxyl group, alkoxy group, and a functional group at terminal bondable to a living molecule, and n represents an integer of 1-20.
